(19) United States
(12) Patent Application Publication
(10) Pub. No.: US 2023/0119141 A1
(43) Pub. Date: Apr. 20, 2023

(54) USES OF TAXIFOLIN FOR RESPIRATORY HEALTH

(71) Applicant: PHYTO TECH CORP., Rancho Santa Margarita, CA (US)

(72) Inventors: Steven CHEN, Coto de Caza, CA (US); Linda Shuo MAY-ZHANG, Rancho Santa Margarita, CA (US)

(21) Appl. No.: 18/047,742

(22) Filed: Oct. 19, 2022

Related U.S. Application Data
(60) Provisional application No. 63/257,406, filed on Oct. 19, 2021.

(51) Int. Cl. A61K 31/352; A61K 31/375; A61K 31/593; A61K 35/60; A61K 33/30; A61K 35/741; A61P 31/14; A61K 31/215; A61K 31/522; A61K 31/513; A61K 31/7068; A61K 31/675; A61K 31/52; A61K 31/7072; A61K 31/536; A61K 31/519; A61K 31/4706; A61K 39/42; A61K 31/4965; A61K 31/7052; A61K 31/7048; A61K 31/635

(52) U.S. Cl. CPC A61K 31/352; A61K 31/375; A61K 31/593; A61K 35/60; A61K 33/30; A61K 35/741; A61P 31/14; A61K 31/215; A61K 31/522; A61K 31/513; A61K 31/7068; A61K 31/675; A61K 31/52; A61K 31/7072; A61K 31/536; A61K 31/519; A61K 31/4706; A61K 39/42; A61K 31/4965; A61K 31/7052; A61K 31/7048; A61K 31/635

(57) ABSTRACT

The present disclosure provides methods for treating or preventing an infectious disease or condition, such as the COVID-19 virus (SARS-CoV-2), by administering a therapeutically effective amount of taxifolin to a subject in need thereof, wherein the taxifolin is administered to the subject in combination with a therapeutically effective amount of one or more additional therapeutic agents.

### BACKGROUND

### Field of the Invention

The present disclosure provides methods of administering a combination of taxifolin and one or more additional therapeutic agents to treat or prevent an infectious disease or condition in a subject in need thereof. In some embodiments, the disclosure provides methods of administering a combination of taxifolin and remdesivir to a subject in need thereof.

### Background

Beginning in December 2019, the emergence of the SARS-CoV-2 virus (i.e., COVID-19) led to a widespread of pneumonia cases throughout the world. Progression of the pneumonia can lead to respiratory failure, and ultimately death. Coronavirus infections pose a major risk to human safety worldwide. Spread of the coronavirus represents a serious source of morbidity and mortality worldwide and a considerable cause of illness and death among people with immunodeficiency associated with aging or different clinical conditions.

The coronavirus consists of spherical, enveloped viruses possessing a single-strand, positive-sense RNA genome ranging from 26 to 32 kilobases in length. The genome of the coronavirus includes a spike protein (S glycoprotein with N-linked glycans) located on the spherical envelope of the virus. The spike protein consists of two units: (1) S1, which mediates the virus binding to receptors on target cells; and (2) S2, which triggers virus and host cell membrane fusion. One example of a cell receptor is angiotensin-converting enzyme 2 (ACE2), which is commonly found along the cell membranes of the lung and has an affinity to interact with S1 unit of the coronavirus. Due to the interaction between the spike protein and the ACE2 receptors, the coronavirus is able to fuse to the ACE2 receptors of the cell membranes in the lung, thereby leading to viral entry into lung cells and replication of the coronavirus. (Serrano et al., Liposomal Lactoferrin as Potential Preventative and Cure for COVID-19, Int'l Journal of Research in Health Sciences, Vol. 8, pp. 8-15, 2020).

There exists a need for new and effective therapeutic strategies to treat infectious diseases or conditions such as COVID-19.

### BRIEF SUMMARY OF THE INVENTION

The present disclosure includes various methods of treating or preventing an infectious disease or condition in a subject in need thereof.

In some embodiments, the method comprises administering a therapeutically effective amount of taxifolin, wherein the taxifolin is administered to the subject in combination with a therapeutically effective amount of one or more additional therapeutic agents.

In some embodiments, the infectious disease or condition is caused by a DNA virus or an RNA virus.

In some embodiments, the RNA virus is a coronavirus.

In some embodiments, the coronavirus is a human coronavirus.

In some embodiments, the human coronavirus is SARS CoV-2.

In some embodiments, the infectious disease or condition is hypercytokinaemia, haemophagocytic lymphohistiocytosis, pneumonia, acute respiratory distress syndrome, or systemic inflammatory response syndrome.

In some embodiments, the one or more additional therapeutic agents comprise an antiviral agent, an antibiotic agent, an antifungal agent, an antiparasitic agent, an antihelminthic drug, or an immune modulator, or a combination thereof

In some embodiments, the one or more additional therapeutic agents comprise one or more antiviral agents.

In some embodiments, the one or more antiviral agents comprise remdesivir.

In some embodiments, the taxifolin is administered in an amount that provides about 10 mg/day to about 1,500 mg/day to the subject.

In some embodiments, the taxifolin is administered to the lung of the subject.

In some embodiments, the taxifolin is administered orally.

In some embodiments, the taxifolin is administered intranasally.

In some embodiments, the taxifolin is administered intratracheally.

### BRIEF DESCRIPTION OF THE DRAWINGS/FIGURES

The accompanying drawings, which are incorporated herein and form a part of the specification, illustrate the present disclosure and, together with the description, further serve to explain the principles thereof and to enable a person skilled in the pertinent art to make and use the same.

FIG. 1 is a schematic illustration of using taxifolin to treat SARS-CoV-2 infection in Golden Syrian hamsters.

FIG. 2 is a line graph showing SARS-CoV-2 viral loads in nasal swabs from animals in the taxifolin and control groups between day 1 and day 7.

FIG. 3 is a line graph showing SARS-CoV-2 viral loads in oral swabs from animals in the taxifolin and control groups between day 1 and day 7.

FIG. 4 is a bar graph showing SARS-CoV-2 viral loads in major organs from animals in the taxifolin and control groups at day 7.

### DETAILED DESCRIPTION OF THE INVENTION

The present disclosure will now be described in detail with reference to embodiments thereof as illustrated in the accompanying drawings, in which like reference numerals are used to indicate identical or functionally similar elements. References to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

All publications, patents, and other references mentioned herein are incorporated by reference in their entireties for all purposes as if each individual publication or patent application were specifically and individually indicated to be incorporated by reference. In addition, citation or identification of any reference in this application shall not be construed as an admission that such reference is available as prior art to the present disclosure.

### Terminology

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular

As used herein and in the appended claims, the singular forms “a,” “an,” and “the” include plural references unless the context clearly dictates otherwise. For example, the term “a compound” or “at least one compound” may include a plurality of compounds, including mixtures thereof. The terms “a”, “an,” “the,” “one or more,” and “at least one,” for example, can be used interchangeably herein.

As used herein, the term “about,” when used to modify an amount related to the disclosure, refers to variation in the numerical quantity that can occur, for example, through routine testing and handling; through inadvertent error in such testing and handling; through differences in the manufacture, source, or purity of ingredients employed in the disclosure; and the like. Whether or not modified by the term “about”, the claims include equivalents of the recited quantities. In some embodiments, the term “about” means plus or minus 10% of the reported numerical value.

Throughout this application, various embodiments of this disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range, such as from 1 to 6 should be considered to have specifically disclosed subranges such as from 1 to 2, from 1 to 3, from 1 to 4, from 1 to 5, from 2 to 3, from 2 to 4, from 2 to 5, from 2 to 6, from 3 to 4, from 3 to 5, from 3 to 6, etc., as well as individual numbers within that range, for example, 1, 2, 3, 4, 5, and 6. This applies regardless of the breadth of the range.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “comprises,” “comprising,” “includes,” “including,” “having,” and their conjugates are interchangeable and mean “including but not limited to.” It is understood that wherever aspects are described herein with the language “comprising,” otherwise analogous aspects described in terms of “consisting of” and/or “consisting essentially of” are also provided.

The term “consisting of” means “including and limited to.”

The term “consisting essentially of” means the specified material of a composition, or the specified steps of a method, and those additional materials or steps that do not materially affect the basic characteristics of the material or method.

A “therapeutically effective amount” or “effective amount” of a composition is a predetermined amount calculated to achieve the desired result. The activity contemplated by the present methods includes both medical therapeutic and/or prophylactic treatment, as appropriate. The specific dose of a compound administered according to this disclosure to obtain therapeutic and/or prophylactic effects will, of course, be determined by the particular circumstances surrounding the case, including, for example, the compound administered, the route of administration, and the condition being treated. A therapeutically effective amount of compound of this disclosure is typically an amount such that when it is administered in a physiologically tolerable excipient composition, it is sufficient. Therapeutically effective amounts may be administered according to a dosing schedule.

The terms “treat,” “treating,” and “treatment” refer to both therapeutic treatment and prophylactic or preventative measures, wherein the object is to prevent or slow down (lessen) an undesired physiological condition, disease, or disorder, or to obtain beneficial or desired physiological results (e.g., clinical, medical, and/or veterinary results). For purposes of this disclosure, beneficial or desired results include, but are not limited to, alleviation or elimination of the symptoms or signs associated with a condition, disease, or disorder; diminishment of the extent of a condition, disease, or disorder; stabilization of a condition, disease, or disorder, (i.e., where the condition, disease, or disorder is not worsening); delay in onset or progression of the condition, disease, or disorder; amelioration of the condition, disease, or disorder; remission (whether partial or total and whether detectable or undetectable) of the condition, disease, or disorder; or enhancement or improvement of a condition, disease, or disorder. Treatment includes eliciting a physiologically significant response without excessive side effects. Treatment also includes prolonging survival as compared to expected survival if not receiving treatment.

As used herein, the terms “prevent” and “preventing” refer to partially or completely delaying onset of an infection, disease, disorder, and/or condition; partially or completely delaying onset of one or more signs, symptoms, features, or manifestations (e.g., clinical or physiological signs, symptoms, features, or manifestations) of a particular infection, disease, disorder, and/or condition; partially or completely delaying progression from an infection, a particular disease, disorder, and/or condition; and/or decreasing the risk of developing a pathology associated with the infection, the disease, disorder, and/or condition.

As used herein, the terms “subject” and “patient” are used interchangeably. The subject can be an animal. In some embodiments, the subject is a mammal such as a non-human animal (e.g., cow, pig, horse, cat, dog, rat, mouse, monkey or other primate, etc.). In some embodiments, the subject is a human.

The term “infection” refers to invasion and multiplication of microorganisms (e.g., bacteria, viruses, fungi, and protozoa).

As used herein, the term “infectious disease or condition” refers to a disease caused by the presence of microorganisms, e.g., a virus, in a subject. Symptoms related to an infectious diseases or conditions include both the direct effects of the virus and the effects on or changes within the body that occur as a result of the subject's body (e.g., its immune system) response to the virus.

The term “human coronavirus” as used herein refers to a positive-stranded RNA virus that has a lipid envelope studded with club-shaped projections that infect humans, and mutated strains thereof. Sexton et al., Journal of Virology 90:7415-7428 (2016).

The terms “HCoV-229E,” “HCoV-0C43,” “HCoV-NL63,” “HCoV-HKUL” “SARS-CoV,” “MERS-CoV,” and “SARS-CoV-2” as used herein refer to the coronavirus pathogens known to infect humans. Lim et al., Diseases 2016, 4, 26; doi:10.3390/diseases4030026; Lai et al., International Journal of Antimicrobial Agent 55:1-9 (2020).

The terms “severe acute respiratory syndrome” or “SARS” as used herein refers to the viral respiratory disease caused by SARS-CoV.

The term “Middle East Respiratory Syndrome” or “MERS” as used herein refers to the viral respiratory disease caused by MERS-CoV.

The term “Coronavirus Disease 2019” or “COVID-19” as used herein refers to the viral respiratory disease caused by SARS-CoV-2.

“Administering” when used in conjunction with a therapeutic means to administer a therapeutic directly into or onto a target tissue or to administer a therapeutic to a patient, whereby the therapeutic positively impacts the tissue to which it is targeted. “Administering” a composition may be accomplished by, for example, inhaling, intravenous, subcutaneous, intramuscular, or intralesional injection, oral administration, topical administration, intranasal administration, intratracheal administration, or by these methods in combination with other known techniques. Such combination techniques can include the use of delivery agents. When more than one different therapeutic agent is administered, the agents may be administered by the same or different routes of administration and/or at the same or differing times. As is understood in the art, an agent can be administered according to a dosing schedule.

Synergy can be expressed in terms of a “Synergy Index (SI),” which generally can be determined by the method described by F. C. Kull et al. Applied Microbiology 9, 538; (1961), from the ratio determined by:

QₐQ_{A}+Q_{b}Q_{B}=Synergy Index (SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

As used herein, the term “stereoisomers” is a general term for all isomers of an individual molecule that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem. 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [a]obs is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography, or optical polarimetry.

Administering Taxifolin and One or More Additional Therapeutic Agents

The present disclosure provides new therapeutic regimens for treating or preventing an infectious disease or condition, such as coronavirus (e.g., HCoV 229E, HCoV 0C43, HCoV NL63, HCoV HKU1, SARS CoV, MERS CoV, or SARS CoV-2), by administering a therapeutically effective amount of taxifolin in combination with a therapeutically effective amount of one or more additional therapeutic agents.

In some embodiments, the infectious disease or condition is caused by a DNA virus.

In another embodiment, the DNA virus is hepatitis B virus, hepatitis C virus, human cytomegalovirus, or herpes simplex virus type 1. In another embodiment, the DNA virus is hepatitis C virus.

In another embodiment, the infectious disease or condition is caused by a RNA virus. In another embodiment, the RNA virus is respiratory syncytial virus, parainfluenza-3 virus, bovine viral diarrhea virus, Venezuelan equine encephalomyelitis virus, Dengue virus, yellow fever virus, Coxsackie B3 virus, encephalomyocarditis virus, influenza A virus, Zika virus, Ebola virus, Junin virus, Lassa Fever virus, Chikungunya virus, or coronavirus.

In another embodiment, the RNA virus is a coronavirus.

In another embodiment, the coronavirus is a human coronavirus, or a mutated strain thereof, or an animal coronavirus, or a mutated strain thereof.

In another embodiment, the coronavirus is a human coronavirus, or a mutated strain thereof.

In another embodiment, the coronavirus is a human coronavirus.

In another embodiment, the coronavirus is a mutated strain of a human coronavirus.

In another embodiment, the coronavirus is an animal coronavirus, or a mutated strain thereof

In another embodiment, the coronavirus is an animal coronavirus.

In another embodiment, the coronavirus is a mutated strain of an animal coronavirus.

In another embodiment, the human coronavirus is HCoV-229E, HCoV-0C43, HCoV-NL63, HCoV-HKU1, SARS-CoV, MERS-CoV, or SARS-CoV-2, or a mutated strain of HCoV-229E, HCoV-0C43, HCoV-NL63, HCoV-HKU1, SARS-CoV, MERS-CoV, or SARS-CoV-2.

In another embodiment, the human coronavirus is HCoV-229E, HCoV-0C43, HCoV-NL63, HCoV-HKU1, SARS-CoV, MERS-CoV, or SARS-CoV-2.

In another embodiment, the human coronavirus is a mutated strain of HCoV-229E, HCoV-0C43, HCoV-NL63, HCoV-HKU1, SARS-CoV, MERS-CoV, or SARS-CoV-2.

In another embodiment, the human coronavirus is HCoV-229E

In another embodiment, the human coronavirus is a mutated strain of HCoV-229E.

In another embodiment, the human coronavirus is HCoV-OC43.

In another embodiment, the human coronavirus is a mutated strain of HCoV-OC43.

In another embodiment, the human coronavirus is HCoV-NL63.

In another embodiment, the human coronavirus is a mutated strain of HCoV-NL63.

In another embodiment, the human coronavirus is HCoV-HKU1.

In another embodiment, the human coronavirus is a mutated strain of HCoV-HKU1.

In another embodiment, the human coronavirus is SARS-CoV.

In another embodiment, the human coronavirus is a mutated strain of SARS-CoV.

In another embodiment, the human coronavirus is MERS-CoV.

In another embodiment, the human coronavirus is a mutated strain of MERS-CoV.

In another embodiment, the human coronavirus is SARS-CoV-2.

In another embodiment, the human coronavirus is a mutated strain of SARS-CoV-2.

In another embodiment, the infectious disease or condition is the common cold, pneumonia, bronchitis, SARS, MERS, or COVID-19.

In another embodiment, the infectious disease or condition is the common cold.

In another embodiment, the infectious disease or condition is pneumonia.

In another embodiment, the infectious disease or condition is bronchitis.

In another embodiment, the infectious disease or condition is SARS.

In another embodiment, the infectious disease or condition is MERS.

In another embodiment, the infectious disease or condition is COVID-19.

In another embodiment, the infectious disease or condition is hypercytokinaemia.

In another embodiment, the infectious disease or condition is haemophagocytic lymphohistiocytosis.

In another embodiment, the infectious disease or condition is acute respiratory distress syndrome.

In another embodiment, the infectious disease or condition is systemic inflammatory response syndrome.

Taxifolin (5,7,3′,4′-flavan-on-ol) is a naturally occurring compound found in a variety of plant-based foods, including fruits, vegetables, and grains. Taxifolin belongs to the subclass flavanonols in the flavonoids. Taxifolin has two stereocenters on the C-ring, thus producing two pairs of enantiomers, including (+)-taxifolin and (−)-taxifolin. Taxifolin may be isolated and purified from natural sources. Taxifolin may be also be produced synthetically. Methods of producing taxifolin are described in, for example, WO 2013113329, and US Pat. No. 10,975,403, and such methods of production are incorporated herein by reference in their entirety.

The term “taxifolin” as used herein refers, collectively, to 2-(3,4-dihydroxyphenyl)-3,5,7-trihydroxychroman-4-one, all possible stereoisomers, e.g., enantiomers and diastereomers, and mixtures, e.g., racemic mixtures, thereof, and the pharmaceutically acceptable salts thereof. The structure of 2-(3,4-dihydroxyphenyl)-3,5,7-trihydroxychroman-4-one is:

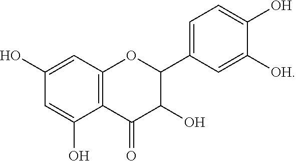 [SMILES: O=C1c2c(O)cc(O)cc2OC(c2ccc(O)c(O)c2)C1O]

The present disclosure encompasses the use of stereoisomers of 2-(3,4-dihydroxyphenyl)-3,5,7-trihydroxychroman-4-one.

In some embodiments, taxifolin is (2R,3R)-2-(3,4-dihydroxyphenyl)-3,5,7-trihydroxychroman-4-one (also known as (2R,3R)-3,3′,4′,5,7-pentahydroxyflavan-4-one; (2R,3R)-trans-dihydroquercetin; (2R,3R)-dihydroquercetin; (+)-dihydroquercetin; (+)-taxifolin), which has the structure shown below:

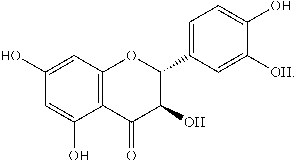 [SMILES: O=C1c2c(O)cc(O)cc2O[C@H](c2ccc(O)c(O)c2)[C@H]1O]

In some embodiments, taxifolin is (2S,3R)-2-(3,4-dihydroxyphenyl)-3,5,7-trihydroxychroman-4-one (also known as (2S,3R)-3,3′,4′,5,7-pentahydroxyflavan-4-one), which has the structure shown below:

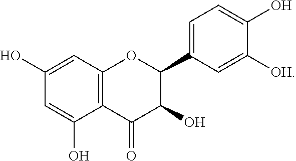 [SMILES: O=C1c2c(O)cc(O)cc2O[C@@H](c2ccc(O)c(O)c2)[C@H]1O]

In some embodiments, taxifolin is (2S,3S)-2-(3,4-dihydroxyphenyl)-3,5,7-trihydroxychroman-4-one (also known as (2S,3S)-3,3′,4′,5,7-pentahydroxyflavan-4-one), which has the structure shown below:

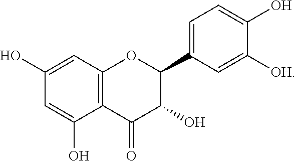 [SMILES: O=C1c2c(O)cc(O)cc2O[C@@H](c2ccc(O)c(O)c2)[C@@H]1O]

In some embodiments, taxifolin is (2R,3S)-3,3′,4′,5,7-pentahydroxyflavan-4-one, which has the structure shown below:

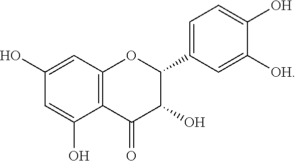 [SMILES: O=C1c2c(O)cc(O)cc2O[C@H](c2ccc(O)c(O)c2)[C@@H]1O]

In some embodiments, taxifolin is a single stereoisomer, e.g., (2R,3R)-2-(3,4-dihydroxyphenyl)-3,5,7-trihydroxychroman-4-one. In some embodiments, taxifolin is a mixture of one or more stereoisomers. In some embodiments, taxifolin is a racemic mixture.

In some embodiments, taxifolin is a single stereoisomer having an enantiomeric excess (ee) of about 50% or more. In another embodiment, the ee is about 55% or more, about 60% or more, about 65% or more, about 70% or more, about 75% or more, about 80% or more, about 85% or more, about 90% or more, about 95% or more, about 98% or more, or about 99% or more. In another embodiment, the ee is about 100%.

In some embodiments of any one of the methods provided herein, the taxifolin in the composition as described herein is substantially purified. In some embodiments of any one of the methods provided herein, the taxifolin in the composition has a chemical purity (as determined by HPLC) greater than about 70%, greater than about 71%, greater than about 72%, greater than about 73%, greater than about 74%, greater than about 75%, greater than about 76%, greater than about 77%, greater than about 78%, greater than about 79%, greater than about 80%, greater than about 81%, greater than about 82%, greater than about 83%, greater than about 84%, greater than about 85%, greater than about 86%, greater than about 87%, greater than about 88%, greater than about 89%, greater than about 90%, greater than about 91%, greater than about 92%, greater than about 93%, greater than about 94%, greater than about 95%, greater than about 96%, greater than about 97%, greater than about 98%, or greater than about 99%, inclusive of all ranges and subranges there between. In some embodiments of any one of the methods provided herein, the taxifolin has a chemical purity of about 95% or more. In some embodiments of any one of the methods or compositions provided herein, the taxifolin in the composition as described herein has a chemical purity of 98% or more, e.g., 99% or more. In some embodiments of any one of the methods provided herein, this purity of the taxifolin refers to the taxifolin that is used in an ultimate product, which ultimate product can include other active and/or inactive agents.

In some embodiments of any one of the methods or compositions provided herein, the taxifolin composition can be in a free-flowing powder form. Without wishing to be bound by theory, the flow of powder can be affected by factors such as shape of the particles, surface of the particles, and the presence of electrostatic charges. In some embodiments of any one of the methods or compositions provided herein, to improve the flowability, flow enhancers may be used in the composition to reduce interparticle friction. In some embodiments of any one of the methods or compositions provided herein, the taxifolin composition can be in any form that is suitable for the methods or compositions as described herein.

In some embodiments, an effective amount of taxifolin is an amount that, when administered in combination therapy, is effective to reduce viral infection, such as an infection by the coronavirus, by at least about 10%, and more preferably at least about 15%, 20%, 25%, 30%, 35%, 40%, 45%, or even at least about 50%, or more, compared with the degree of the viral infection in the individual prior to treatment with the combination therapy. In certain embodiments, the taxifolin is administered according to a dosing schedule, and when administered in a combination therapy, is effective to reduce viral infection, particularly infection by the coronavirus, by at least about 10%, and more preferably at least about 15%, 20%, 25%, 30%, 35%, 40%, 45%, or even at least about 50%, or more, compared with the degree of viral infection in the individual prior to treatment with the combination therapy.

In some embodiments, the disclosure provides methods of treating a viral infection, such as infection by coronavirus, that include administering a therapeutically effective amount of taxifolin to a patient according to a dosing regimen (e.g., a dose and dosing schedule) and/or dosing schedule effective to ameliorate one or more symptoms of viral infection. In some embodiments, the subject (such as a human) may be treated by administering a therapeutically effective amount (e.g., a single dose) of taxifolin and repeating the step of administering the therapeutically effective amount of taxifolin at a predetermined time interval over a predetermined amount of time. For example, taxifolin can be administered as a dry powder, and the therapeutically effective amount (e.g., a single dose) can be from about 10 mg to about 1500 mg of taxifolin, or from about 10 mg to about 750 mg, or from about 20 mg to about 900 mg, or from about 100 mg to 1400 mg, or from about 300 mg to about 1000 mg of taxifolin, about 500 mg to about 1000 mg of taxifolin, or about 750 mg to about 1000 mg of taxifolin. In some embodiments, taxifolin can be administered as a powder dissolved in a liquid carrier, such as, for example, saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure. In some embodiments, the concentration of taxifolin per saline can be from about 10 μg/ml to 1000 μg/ml, such as, for example, from about 100 μg/ml to 800 μg/ml.

The step of administering the therapeutically effective amount of taxifolin can be repeated at a predetermined time interval. In some embodiments, the predetermined time interval can be from about three hours to twenty-four hours, or from about four hours to twelve hours, or from about six hours to about twelve hours, or from about six hours to about eight hours. In some embodiments, the step of administering the therapeutically effective amount of taxifolin at a predetermined time interval can be repeated over a predetermined amount of time, such as, for example, repeating from about 1 day to about one month, or repeating over four weeks, or repeating over three weeks, or repeating over two weeks, or repeating over ten days, or repeating over 5 days.

The amount of taxifolin required to achieve a particular outcome or effect, may vary based on several factors including, but not limited to: the route of administration, the body weight of the subject in need thereof, the level of taxifolin required to achieve a desired effect, the stability of the taxifolin, etc. One of skill in the art can readily determine an amount of taxifolin to provide or administer to a subject for any one of the purposes provided herein, as relevant and needed. In some embodiments of any one of the methods provided herein, the taxifolin can be in any amount suitable for human subjects in need thereof as described herein.

In some embodiments, the taxifolin may be in a capsule (e.g., aerosol carrier) and/or a unit dosage form. In some embodiments, taxifolin is administered by inhalation or insufflation (through either the mouth or nose). For example, taxifolin can be administered by using a dry powder inhaler, a meter dose inhaler, a nebulizer (e.g., jet, ultrasonic, or mesh), or any other suitable vaping device.

In some embodiments, taxifolin can be administered by inhaling an aerosol containing a therapeutically effective amount of taxifolin. In some embodiments, the aerosol includes solid or liquid particles of taxifolin suspended in a gas. In some embodiments, the taxifolin is encapsulated in an aerosol carrier. In some embodiments, the aerosol carrier can include particles having an aerodynamic mean diameter in a range from about 1μm to about 5μm (e.g., from about 1μm to about 4μm, or from about 1 p.m to about 3 p.m, or from about 1μm to about 2μm, or from about 2μm to about 4μm, or from about 2μm to about 3 p.m) so that the aerosol carrier can effectively deliver the taxifolin to the respiratory tract (e.g., trachea, bronchi, bronchioles, or alveoli) without first dissolving or being absorbed. In some embodiments, the aerosol carrier can include at least one of a liposome, a surfactant, gelatin, a plurality of solid lipid nanoparticles, a mucinolytic enzyme, and a microparticle. In some embodiments, the aerosol carrier can be provided as a solid (e.g., dry powder) or a liquid (e.g., aqueous solution) suitable for entrapping taxifolin for air suspension. For example, taxifolin can be formulated for nebulization by being stabilized with polyethylene glycols or an excipient, such as, for example, buffers, sugars, polyols, and/or amino acids.

In some embodiments, the aerosol carrier can include a liposome that includes an aqueous core surrounded by a lipid bilayer possessing both hydrophilic and hydrophobic properties. In some embodiments, types of liposomes used to encapsulate taxifolin can include cyclosporin A (CsA)-dilauroylphosphatidylcholine (DLPC) liposomes, hydrogenated soy phosphatidylcholine (HSPC) liposomes, and liposomal ciprofloxacin. In some embodiments, liposome aerosol carriers can prepared by known methods, such as with phospholipids (e.g., egg phosphatidylcholine (PC), distearoyl phosphatidylcholine (DSPC) and dipalmitoylphosphatidylcholine (DPPC)). In some embodiments, the liposome is made into a solution suitable for aerosolization, using a pharmaceutically acceptable excipient, such as water, aqueous saline (with or without buffer), dextrose and water, or other known substances.

In some embodiments, the aerosol carrier can include a surfactant, such as, for example, micelle sodium, lauryl sulphate, polysorbates, Tween 20, Tween 80 and Tween 90 solutions and sorbitan trioleate dispersions. For example, taxifolin can be trapped in a core of micelle that forms a hydrophilic shell to protect the taxifolin from being dissolved when passing through the airway of the patient.

In some embodiments, the aerosol carrier includes a microparticle having a median diameter in a range from about 1 μm to about 999 μm. In some embodiments, a microparticle can be formed as a microsphere having a uniform sphere comprised of a polymeric matrix. In some embodiments, a microparticle can be formed as a microcapsule container comprised of an oily core surrounded by a thin polymeric membrane.

In some embodiments, any suitable inhalation device, such as, for example, a nebulizer, a dry powder inhaler, metered dose inhaler, and/or a sprayer, can be used to generate the aerosol containing the therapeutically effective amount of taxifolin. In some embodiments, the inhalation device can be a nebulizer to convert taxifolin dissolved in a liquid solution to a mist stream suitable for inhalation. In some embodiments, the nebulizer can be a jet nebulizer, an ultrasonic nebulizer, or a mesh nebulizer. In some embodiments, a sprayer having the combination of a high-pressure (e.g., operating up to 3000 psi) syringe and a delivery tube coupled to a discharge end of the syringe (e.g., PennCentury™ MicroSprayer) can be used to generate a plume of aerosol containing taxifolin in liquid form that can be administered directly into the respiratory tract of a user. In some embodiments, a nebulizer is selected to produce sufficiently small particles (e.g., producing particles with a mean particle diameter of less than about 5.0 μm, such as a mean particle diameter of about 1.0 μm to about 3.0 μm, that ensure effective delivery to the lung (e.g., alveoli). In some embodiments, when the taxifolin is supplied as a dry powder, the inhalation device can be an inhaler, such as a dry powder inhaler or a metered dose inhaler.

The taxifolin composition described herein can be used as or mixed with dietary supplements, medical compositions, cosmeceuticals, for nutrition, as well as in pharmaceutical products.

In some embodiments of any one of the methods or compositions provided herein, the taxifolin composition can be formulated or prepared as a food supplement for oral consumption. In some embodiments of any one of the methods or compositions provided herein, the taxifolin composition can be formulated or prepared for dietary products, such as food products, medical foods, etc. In some embodiments of any one of the methods or compositions provided herein, the taxifolin composition can be formulated or prepared for beverages.

Food compositions according to the disclosure include any preparations or compositions which are suitable for consumption and are used for nutrition or enjoyment purposes. They are generally products which are intended to be eaten by humans or animals and introduced into the body through the mouth, to remain there for a certain time and then either be eaten (e.g., ready-to-eat foodstuffs or feeds, see also herein below) or removed (e.g. chewing gums). Such products include any substances or products which in the processed, partially processed or unprocessed state are to be ingested by humans or animals. They also include substances which are added to orally consumable products during their manufacture, preparation or treatment and which are intended to be introduced into the human or animal oral cavity.

The food compositions according to the disclosure also include substances which in the unchanged, treated or prepared state are to be swallowed by a human or animal and then digested; in this respect, the orally consumable products according to the disclosure also include casings, coatings or other encapsulations which are to be swallowed at the same time or which may be expected to be swallowed. The expression “food composition” covers ready-to-eat foodstuffs, beverages and feeds, that is to say foodstuffs, beverages or feeds that are already complete in terms of the substances that are important for the taste. The expressions “ready-to-eat foodstuff” and “ready-to-eat feed” also include drinks as well as solid or semi-solid ready-to-eat foodstuffs or feeds. Examples which may be mentioned are frozen products, which must be thawed and heated to eating temperature before they are eaten. Products such as yoghurt or ice-cream as well as chewing gums or hard caramels are also included among the ready-to-eat foodstuffs or feeds of the current disclosure.

Non-limiting examples of food and beverage products include carbonated soft drinks, ready to drink beverages, energy drinks, isotonic drinks, low-calorie drinks, zero-calorie drinks, sports drinks, teas, fruit and vegetable juices, juice drinks, dairy drinks, yoghurt drinks, alcohol beverages, powdered beverages, bakery products, cookies, biscuits, baking mixes, cereals, confectioneries, candies, toffees, chewing gum, dairy products, flavored milk, yoghurts, flavored yoghurts, cultured milk, soy sauce and other soy base products, salad dressings, mayonnaise, vinegar, frozen-desserts, meat products, fish-meat products, bottled and canned foods, tabletop sweeteners, fruits and vegetables.

The compositions can be used “as-is” or in combination with sweeteners, flavors and food ingredients. Dry compositions, such as powders, granules or tablets are stable indefinitely when stored under dry conditions at room temperature. Compositions in the form of aqueous solutions are stable indefinitely when frozen. If a preservative such as benzoic acid or its salts, sulphur dioxide or sodium meta-bisulphite is added to such a composition, it may be stored almost indefinitely at room temperature.

Non-limiting examples of flavors include lemon, orange, fruity, banana, grape, pear, pineapple, bitter almond, cola, cinnamon, sugar, cotton candy, vanilla flavors.

Non-limiting examples of other food ingredients include flavors, acidulants, organic and amino acids, coloring agents, bulking agents, modified starches, gums, texturizers, preservatives, antioxidants, emulsifiers, stabilizers, thickeners, gelling agents.

Aspects of the present disclosure provide, in some embodiments, a composition comprising a therapeutically effective amount of taxifolin. The composition can be a pharmaceutical composition. In some embodiments of any one of the methods or compositions provided herein, the pharmaceutical composition can include one or more pharmaceutically acceptable carriers.

In some embodiments of any one of the methods or compositions provided herein, the pharmaceutical composition comprising a therapeutically effective amount of taxifolin can comprise other pharmaceutical ingredients, such as preservatives, or chemical stabilizers.

Formulation of pharmaceutically-acceptable excipients and carrier solutions is known to those of skill in the art, as is the development of suitable dosing and treatment regimens for using the particular compositions described herein. The amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such formulations, and as such, a variety of dosages and treatment regimens may be desirable.

The forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations can contain a preservative to prevent the growth of microorganisms. In many cases the form is sterile and fluid to the extent that easy syringeability exists. It may be stable under the conditions of manufacture and storage and preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Routes of administration are known to those of skill in the art and may be combined, if desired.

Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is known in the art. Supplementary active ingredients can also be incorporated into the compositions. The phrase “pharmaceutically-acceptable” refers to molecular entities and compositions that do not produce an allergic or similar untoward reaction when administered to a host.

Purified taxifolin can have purity of about 98%, 99% or more, or any other purity level that is suitable for the methods and the formulating of the compositions as disclosed herein.

Suitable carriers may be readily selected by one of skill in the art in view of the type of ultimate product and agents. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin. In some embodiments of any one of the methods or compositions provided herein, the composition can comprise flavoring agents or sweeteners.

Methods described herein comprise providing or administering the composition in sufficient amounts for any one of the endpoints provided herein without undue adverse effects.

The taxifolin composition can be formulated or prepared in a neutral or salt form.

In some embodiments, the one or more additional therapeutic agents comprise an antiviral agent, an antibiotic agent, an antifungal agent, an antiparasitic agent, an antihelminthic drug, or an immune modulator, or a combination thereof

Non-limiting exemplary immune modulators include beta interferon, alpha interferon, phosphodiesterase 4 inhibitors, corticosteroids, e.g., dexamethasone, and prostaglandin synthetase inhibitors.

In some embodiments, the one or more additional therapeutic agents comprise one or more antiviral agents.

In some embodiments, the one or more antiviral agents comprise one or more reverse transcriptase inhibitors (RTIs). In some embodiments, the one or more RTIs comprise abacavir (ZIAGEN™), abacavir/lamivudine (Epzicom), abacavir/lamivudine/zidovudine (TRIZIVIR™), adefovir, alovudine, amdoxovir, apricitabine, ATRIPLA®, BARACLUDE®, BIKTARVY®, censavudine, COVIRACIL™, DAPD/DXG, D-D4FC, dexelvucitabine, didanosine (VIDEX™), didanosine extended-release (Videx EC), dOTC, islatravir, emtricitabine (EMTRIVA™), emtricitabine/tenofovir alafenamide)(DESCOVY®), emtricitabine/tenofovir disoproxil fumarate (TRUVADA), elvucitabine, fosalvudine, lamivudine/zidovudine (COMBIVIR™), EVIPLERA™, GENVOYA®, HIVID™, KIVEXA™, lamivudine (EPIVIR™), LODENOSINE™, ODEFSEY®, PREVEON®, racivir, stampidine, stavudine (ZERIT™), STRIBILD®, TENOFOVIR™, tenofovir disoproxil fumarate (VIREAD™), TRIUMEQ®, Trizivir, VEMLIDY , zidovudine (RETROVIR™), delavirdine, efavirenz, etravirine, nevirapine, or rilvipirine.

In some embodiments, the one or more antiviral agents comprise oseltamivir, ganciclovir, lopinavir/ritonavir (Kaletra®), molnupiravir, or remdesivir.

In some embodiments, the one or more antiviral agents comprise molnupiravir.

In some embodiments, the one or more antiviral agents comprise remdesivir.

In some embodiments, the one or more additional therapeutic agents comprise

Tocilizumab (Actemra®), Favipiravir (Avigan®), Tocilizumab/favipiravir, Leronlimab (PRO 140), Remdesivir, Ruxolitinib (Kevzara®), Sarilumab, Chloroquine phosphate (Aralen®, Resochin®), Chloroquine hydrochloride, Azithromycin (Zithromax®), hydroxychloroquine sulfate/azithromycin, Lopinavir/Ritonavir (Kaletra®), Eculizumab (Soliris®), Human monoclonal antibody targeting SARS-CoV-2, APN01, Danoprevir (Ganovo®), TJM2 (TJ003234), Selinexor (XPOVIO®), Remestemcel-L (RYONCIL™) LAM-002 (apilimod), Rintatolimod (Ampligen®), DAS181, CM4620-IE, CAP-1002, SAB-185, ENU200, Camostat mesylate, IFX-1, Namilumab (IZN-101), GIAPREZA™ (angiotensin II), MN-166 (ibudilast), Rebif® (interferon beta-1a), Ivermectin (Stromectol®, Mectizan®), NVX-CoV2373, Thiolanox®, Plitidepsin (Aplidin®), Opaganib (Yeliva®), RHB-107, Opaganib/RHB-107, EIDD-2801, Gimsilumab, TAK-888, ARMS-1, GENOSYL® (nitric oxide) gas, INOpulse®, BPI-002, rhu-pGSN, Galidesivir (BCX4430), BXT-10, L-glutamine oral powder (Endari®), Sylvant (siltuximab), Linebacker, Equivir, HTCC (N-(2-hydroxypropyl)-3-trimethylammonium 47 chitosan chloride), Darunavir (Prezista®), Darunavir/cobicistat (Prezcobix™) INOmax® (nitric oxide), WP1122, OYA1, Arbidol (umifenovir), Remescor®, MAN-01, STI-4920 (CMAB020), TZLS-501, IFN-alpha2b, Niclosamide, KL4, or WP1122, or a combination thereof.

In some embodiments, the taxifolin is administered to the subject before the one or more additional therapeutic agents.

In some embodiments, the taxifolin is administered to the subject after the one or more additional therapeutic agents.

In some embodiments, the taxifolin is administered to the subject at the same time as the one or more additional therapeutic agents.

The foregoing description of the specific embodiments will so fully reveal the general nature of the invention(s) that others can, by applying knowledge within the skill of the art, readily modify and/or adapt for various applications such specific embodiments, without undue experimentation, without departing from the general concept of the present invention(s). Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The following examples are illustrative, but not limiting, of the present inventions.

Other suitable modifications and adaptations of the variety of conditions and parameters normally encountered in the field, and which would be apparent to those skilled in the art, are within the spirit and scope of the inventions.

### EXAMPLES

### Example 1

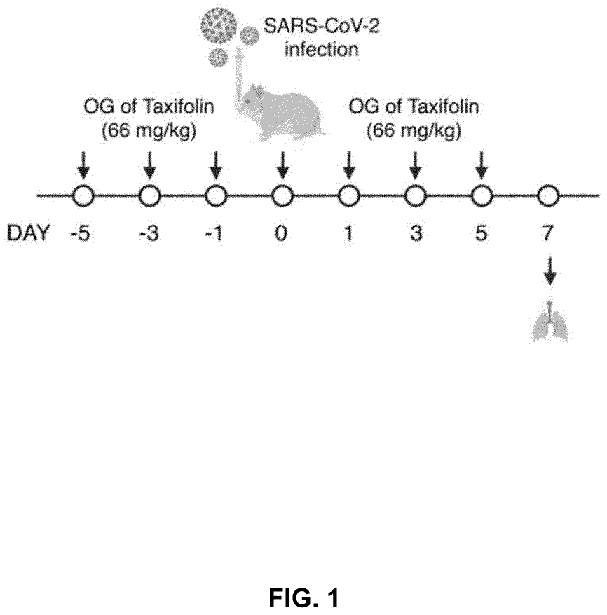

The therapeutic efficacy of taxifolin against SARS-CoV-2 infection was examined in Golden Syrian hamsters, a small animal model that most closely recapitulates clinical and pathological features of COVID-19 in humans. See, e.g., Imai, M, et al., Proc. Nat. Acad. Sci. 117(28):16587-16595 (2020); Reagan-Shaw, S., et al., FASEB J. 22:659-661 (2007). As illustrated in FIG. 1, animals were divided into two groups and intranasally inoculated with SARS-CoV-2 at day 0. Animals in the taxifolin group (n=15) received prophylactic treatment of taxifolin (66.7 mg/kg) through oral gavage (OG) before the viral infection.

After SARS-CoV-2 inoculation, animals in the taxifolin group were orally given taxifolin (66.7 mg/kg) at days 1, 3, and 5. Animals in the control group (n=10) received an equal amount of PBS through oral gavage. At day 7, certain animals in the taxifolin (n=6) group and control group (n=5) were sacrificed, and the viral loads in major organs were tested.

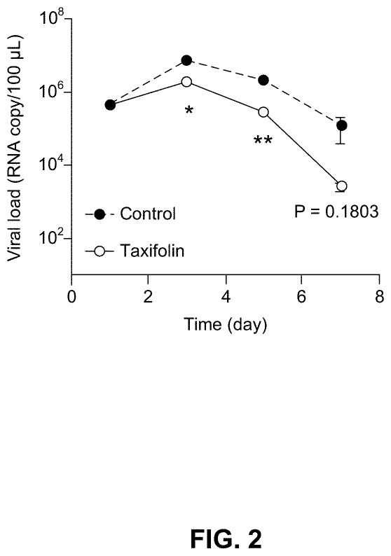

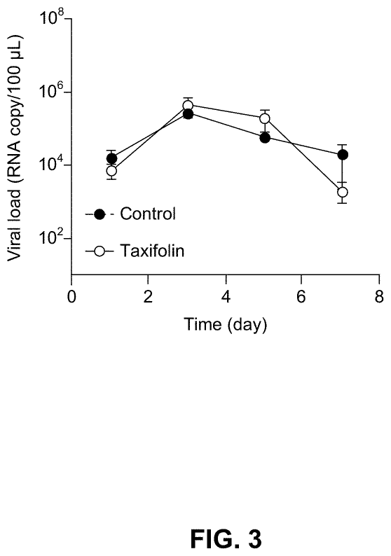

FIG. 2 and FIG. 3 show the viral loads in nasal and oral swabs, respectively, from animals in both groups. Treatment with taxifolin significantly lowered the viral loads in nasal swabs at days 3 and 5 after viral infection. Specifically, animals in the taxifolin group (n=8) showed a viral load of ˜7×10⁴ RNA copies/100 μL at day 5. Animals in control group (n=8) showed a viral load of ˜3×10⁶ RNA copies/100 μL at day 5. Taxifolin also decreased the viral loads in oral swabs at day 7 (FIG. 3).

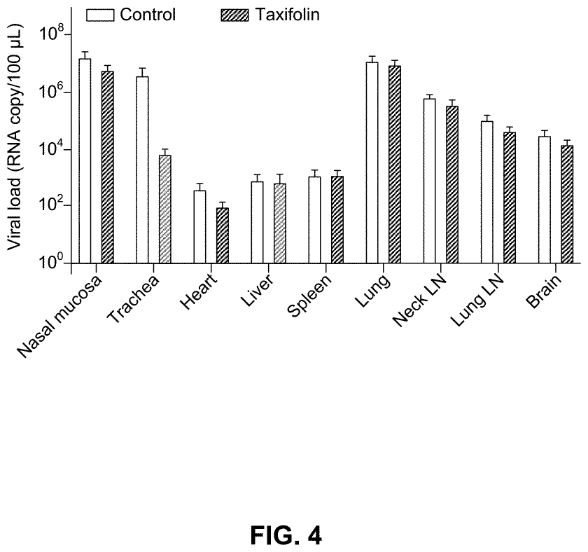

The viral loads in major organs at day 7 (FIG. 4) were determined. Relatively high viral loads were detected from the nasal mucosa in all animals (n=4 for each group), indicating the successful viral infection in the golden hamster model. The treatment of taxifolin clearly reduced the viral loads in the trachea, a major organ that relates to severe pneumonia after SARS-CoV-2 infection in patients.

The breadth and scope of the present invention should not be limited by any of the above-described exemplary embodiments, but should be defined only in accordance with the following claims and their equivalents.

1. A method of treating or preventing an infectious disease or condition in a subject in need thereof, the method comprising administering a therapeutically effective amount of taxifolin, wherein the taxifolin is administered to the subject in combination with a therapeutically effective amount of one or more additional therapeutic agents.

2. The method of claim 1, wherein the infectious disease or condition is caused by a DNA virus or an RNA virus.

3. The method of claim 2, wherein the infectious disease or condition is caused by a DNA virus, wherein the DNA virus is hepatitis B virus, hepatitis C virus, human cytomegalovirus, or herpes simplex virus type 1.

4.-5. (canceled)

6. The method of claim 2, wherein the infectious disease or condition is caused by an RNA virus, wherein the RNA virus is respiratory syncytial virus, parainfluenza-3 virus, bovine viral diarrhea virus, Venezuelan equine encephalomyelitis virus, Dengue virus, yellow fever virus, Coxsackie B3 virus, encephalomyocarditis virus, influenza A virus, Zika virus, Ebola virus, Junin virus, Lassa Fever virus, Chikungunya virus, or a coronavirus.

7. The method of claim 6, wherein the RNA virus is a coronavirus.

8. The method of claim 7, wherein the coronavirus is SARS CoV-2, or a mutated strain thereof.

9.-11. (canceled)

12. The method of claim 1, wherein the infectious disease or condition is hypercytokinaemia, haemophagocytic lymphohistiocytosis, pneumonia, acute respiratory distress syndrome, or systemic inflammatory response syndrome.

13. (canceled)

14. The method of claim 1, wherein the one or more additional therapeutic agents comprise an antiviral agent, an antibiotic agent, an antifungal agent, an antiparasitic agent, an antihelminthic drug, or an immune modulator, or a combination thereof.

15. The method of claim 14, wherein the one or more additional therapeutic agents comprise one or more antiviral agents.

16. The method of claim 15, wherein the one or more antiviral agents comprise one or more reverse transcriptase inhibitors (RTIs).

17. The method of claim 16, wherein the one or more RTIs comprise abacavir (ZIAGEN™), abacavir/lamivudine (Epzicom), abacavir/lamivudine/zidovudine (TRIZIVIR™), adefovir, alovudine, amdoxovir, apricitabine, ATRIPLA®, BARACLUDE®, BIKTARVY®, censavudine, COVIRACIL™, DAPD/DXG, D-D4FC, dexelvucitabine, didanosine (VIDEX™), didanosine extended-release (Videx EC), dOTC, islatravir, emtricitabine (EMTRIVA™), emtricitabine/tenofovir alafenamide)(DESCOVY®), emtricitabine/tenofovir disoproxil fumarate)(TRUVADA®), elvucitabine, fosalvudine, lamivudine/zidovudine (COMBIVIR™), EVIPLERA™, GENVOYA®, HIVID™, KIVEXA™, lamivudine (EPIVIR™), LODENOSINE™, ODEFSEY®, PREVEON®, racivir, stampidine, stavudine (ZERIT™), STRIBILD®, TENOFOVIR™, tenofovir disoproxil fumarate (VIREAD™), TRIUMEQ®, Trizivir, VEMLIDY®, zidovudine (RETROVIR™), delavirdine, efavirenz, etravirine, nevirapine, or rilvipirine.

18. The method of claim 15, wherein the one or more antiviral agents comprise oseltamivir, ganciclovir, lopinavir/ritonavir (Kaletra®), molnupiravir, or remdesivir.

19. The method of claim 1, wherein the one or more additional therapeutic agents comprise Tocilizumab (Actemra®), Favipiravir (Avigan®), Tocilizumab/favipiravir, Leronlimab (PRO 140), Remdesivir, Ruxolitinib (Kevzara®), Sarilumab, Chloroquine phosphate (Aralen®, Resochin®), Chloroquine hydrochloride, Azithromycin (Zithromax®), hydroxychloroquine sulfate/azithromycin, Lopinavir/Ritonavir (Kaletra®), Eculizumab (Soliris®), Human monoclonal antibody targeting SARS-CoV-2, APN01, Danoprevir (Ganovo®), TJM2 (TJ003234), Selinexor (XPOVIO®), Remestemcel-L (RYONCIL™), LAM-002 (apilimod), Rintatolimod (Ampligen®), DAS181, CM4620-IE, CAP-1002, SAB-185, ENU200, Camostat mesylate, IFX-1, Namilumab (IZN-101), GIAPREZATM (angiotensin II), MN-166 (ibudilast), Rebif® (interferon beta-1a), Ivermectin (Stromectol®, Mectizan®), NVX-CoV2373, Thiolanox®, Plitidepsin (Aplidin®), Opaganib (Yeliva®), RHB-107, Opaganib/RHB-107, EIDD-2801, Gimsilumab, TAK-888, ARMS-1, GENOSYL® (nitric oxide) gas, INOpulse®, BPI-002, rhu-pGSN, Galidesivir (BCX4430), BXT-10, L-glutamine oral powder (Endari®), Sylvant (siltuximab), Linebacker, Equivir, HTCC (N-(2-hydroxypropyl)-3-trimethylammonium 47 chitosan chloride), Darunavir (Prezista®), Darunavir/cobicistat (Prezcobix™), INOmax® (nitric oxide), WP1122, OYA1, Arbidol (umifenovir), Remescor®, MAN-01, STI-4920 (CMAB020), TZLS-501, IFN-alpha2b, Niclosamide, KL4, or WP1122, or a combination thereof.

20.-22. (canceled)

23. The method of claim 1, wherein the taxifolin has a chemical purity of at least about 98%.

24. The method of claim 1, wherein the taxifolin is administered in an amount that provides about 10 mg/day to about 1,500 mg/day to the subject.

25.-28. (canceled)

29. The method of claim 1, wherein the taxifolin is administered as a pharmaceutical composition.

30. The method of claim 1, wherein the taxifolin is administered to the lung of the subject.

31. The method of claim 30, wherein the taxifolin is administered by inhaling an aerosol comprising the therapeutically effective amount of taxifolin.

32. (canceled)

33. The method of claim 31, wherein the taxifolin is administered using a dry powder inhaler to generate the aerosol comprising the therapeutically effective amount of taxifolin.

34.-36. (canceled)

37. The method of claim 30, wherein the taxifolin is formulated as a dry powder.

38. The method of claim 30, wherein the taxifolin is formulated as a liquid solution.

39. The method of claim 1, wherein administering the therapeutically effective amount of taxifolin is repeated at a predetermined time interval.

40. (canceled)

41. The method of claim 1, wherein the taxifolin is administered orally.

42. The method of claim 41, wherein the taxifolin is administered as an orally consumable product, wherein the orally consumable product is a dietary capsule or tablet, a sachet, a functional beverage, gummies, chewables, or a nutritional bar.

43.-46. (canceled)

47. The method of claim 1, wherein the taxifolin is administered intranasally.

48. The method of claim 1, wherein the taxifolin is administered intratracheally.

49. The method of claim 1, wherein the taxifolin is administered one, two, or three times per day.

50.-51. (canceled)

52. The method of claim 1 further comprising administering vitamin C, vitamin D, fish oil, zinc, a botanical extract, a probiotic, a prebiotic, or a postbiotic, or a combination thereof to the subject.

53. The method of claim 1, wherein the taxifolin is (2R,3R)-2-(3,4-dihydroxyphenyl)-3,5,7-trihydroxychroman-4-one.

54. The method of claim 53, wherein the (2R,3R)-2-(3,4-dihydroxyphenyl)-3,5,7-trihydroxychroman-4-one has an enantiomeric excess of about 95% or more.
